(19) United States
(12) Patent Application Publication
(10) Pub. No.: US 2022/0105244 A1
(43) Pub. Date: Apr. 7, 2022

(54) METHODS AND COMPOSITIONS FOR PROTECTION OF BIOPROSTHETIC HEART VALVE TISSUE FROM GLYCATION AND ASSOCIATED PROTEIN INCORPORATION

(71) Applicant: The Trustees of Columbia University in the City of New York, New York, NY (US)

(72) Inventors: Giovanni Ferrari, Hastings on Hudson, NY (US); Antonio Frasca, Bronx, NY (US)

(21) Appl. No.: 17/499,636

(22) Filed: Oct. 12, 2021

Related U.S. Application Data
(63) Continuation of application No. PCT/US20/27698, filed on Apr. 10, 2020.
(60) Provisional application No. 62/972,724, filed on Feb. 11, 2020.
(60) Provisional application No. 62/860,586, filed on Jun. 12, 2019.
(60) Provisional application No. 62/833,450, filed on Apr. 12, 2019.

(51) Int. Cl. A61L 27/36; A61K 31/4015; A61K 31/4164

(52) U.S. Cl. CPC A61L 27/3687; A61K 31/4015; A61L 2300/40; A61L 2430/20; A61L 2300/204; A61K 31/4164

(57) ABSTRACT

Compositions and methods for protecting BHV from SVD mechanisms, including non-calcific SVD mechanisms, are provided.

### CROSS-REFERENCE TO RELATED APPLICATIONS

The present application is a Continuation of International Application No. PCT/US20/27698 filed 10 Apr. 2020; which claims the benefit of U.S. Provisional Application Ser. No. 62/972,724 filed 11 Feb. 2020, 62/860,586 filed 12 Jun. 2019, and 62/833,450 filed 12 Apr. 2019; each of which are incorporated herein by reference in its entirety.

### STATEMENT REGARDING FEDERALLY SPONSORED RESEARCH OR DEVELOPMENT

This invention was made with government support under HL122805, HL143008 and HL007343 awarded by the National Institutes of Health. The government has certain rights in the invention.

### MATERIAL INCORPORATED-BY-REFERENCE

Not Applicable.

### BACKGROUND OF THE INVENTION

Heart valve disease is a common disorder, affecting millions worldwide, and at present cannot be treated medically. Surgery is the only option for severe heart valve disease, and can involve either repair or more commonly the use of a prosthetic heart valve. Bioprosthetic heart valves (BHV) fabricated from glutaraldehyde crosslinked heterografts, such as bovine pericardium (BP) or porcine aortic valves (PAV), are widely used to treat severe heart valve disease. BHV are preferred to mechanical prostheses because BHV in general have a lower risk of thrombo-embolic events, and BHV patients typically do not require anticoagulatnts. BHV at this time are either surgically implanted or deployed with transcatheter techniques. Surgical BHV are known to deteriorate after 10-15 years post-operation, most often due to structural valvular degeneration (SVD), frequently involving calcification. SVD occurs regardless of the heterograft material, requiring prosthesis replacement.

Calcification is by far the best established mechanism associated with SVD, wherein it is considered to contribute to tissue stiffening and functional stenosis as well as regurgitation due to gross leaflet deformation. Roughly 70-75% of SVD cases are considered calcific. Non-calcific SVD mechanisms, which account for an estimated 25% or more of BHV failures, have been investigated to a dramatically lesser extent.

Compositions and methods for protecting BHV from SVD mechanisms, including non-calcific SVD mechanisms, would thus represent an important advancement in the field.

### SUMMARY OF THE INVENTION

The present disclosure provides a method for pre-treatment of bioprosthetic tissues. Such bioprosthetic tissues can be used in a number of applications, including: 1) tissues for use in cardiothoracic surgery (bioprosthetic heart valves, both surgical and transcatheter valve prostheses—such bioprosthetic heart valves include stented valves (porcine and pericardial), stentless valves (glutaraldehyde-fixed bovine, porcine, and equine tissues), aortic homografts, and transcatheter valve prostheses (which are mostly bovine and calf pericardium), bovine pericardial patches used in cardiovascular surgery and heart valve repairs (mostly on aortic and mitral reconstruction), Carpentier's Total Artificial Heart, and ventricular aneurysm repair); 2) tissues for use in vascular surgery such as arterial reconstruction and aortic reconstruction; 3) tissues for thoracic surgery, such as glutaraldehyde-fixed porcine pulmonary ligament; 4) tissues for general surgery, for example to repair abdominal hernias, reconstruction of incisional hernias, repair of extrahepatic bile duct strictures, abdominal wall reconstruction, and pelvic floor reconstruction; 5) tissues for urology, such as urethral patch used in laboratory animal studies; 6) tissues for ophthalmology, such as use of bovine pericardium in the treatment of a large corneal perforation secondary to alkali injury; 7) tissues for use in neurosurgery, for example a dural graft; and 8) tissues for use in dentistry, for example, a graft or patch to correct maxillary sinus perforations, maxillary sinus perforations, deep intrabone defects, and periodontal defects.

The methods for pre-treatment of bioprosthetic tissues, such as bioprosthetic heart valve biomaterials and related biomaterials, such as bovine pericardial patches and conduits, are used as a manufacturing step to render the bioprosthetic tissues resistant or inert to physiologic glycation and the covalent incorporation of infiltrated circulating proteins, including, but not limited to, serum albumin, during clinical implantation. In certain embodiments, the method involves the general chemical modification of glycation-susceptible sites in bioprosthetic tissue to generate stable, permanent adducts that act as protecting groups against physiologic glycation. In certain embodiments the method involves the treatment of BHV biomaterial (exemplified by glutaraldehyde-fixed bovine pericardium) with small molecule compounds designed to exhibit specific properties derived from key principles of the method. Specific properties of the exemplary molecules pertaining to this disclosure and the guiding principles of the related method of this disclosure are as follows: 1. The molecules are designed to react directly or indirectly with glycation-susceptible sites as described in order to generate stable adducts that prevent these sites from reacting with physiologic glycation precursors, effectively acting as protecting groups against glycation. These sites are nucleophilic chemical groups in tissue protein amino acid (primarily nitrogenous groups, most prominently including lysine and arginine side chain termini) side chains and N-termini. These groups are prone to spontaneous reaction with sugar-derived aldehyde and ketone groups, which is the initiating step of glycation. Therefore, in more specific embodiments: 2. The molecules are designed to have similar reactivity with glycation-susceptible sites in biomaterial tissue as physiologic glycation precursors, thereby mooting the complexity of the broad chemistry of glycation precursor molecules by reacting with similar sites in general. In other words, they employ aldehydes, ketones, or alternative moieties with similar ultimate reactivity with glycation-susceptible nucleophilic groups as the conjugation activity; 3. Subsequent to glycation-like reaction of the presently disclosed compounds' reactive groups with glycation sites, the compounds are designed to undergo chemical rearrangements that allow for the resolution of intermediates (such as Schiff bases) to stable, permanent adducts that are unreactive or minimally reactive to glycation and do not follow glycation-like chemical rearrangements (e.g., Amadori rearrangement) that could result in the generation of glycation product-like moieties; 4. The primary adducts generated by these anti-glycation pre-treatment molecules are minimally deleterious or non-deleterious to protein function, being relatively small in size, bearing no capability for cross-linking, bearing relatively similar chemical character to the natural side chains being modified (save for their non-reactivity to glycation), and possibly coordinating water molecules naturally resident in collagen fibril void spaces to mask any side chain polarity and molecular shape differences. The present disclosure also specifies the design of two particular small molecule classes (with 1,3-dihydro-2H-pyrrol-2-one and 4-(1H-Imidazol-4-yl)butanal as scaffolds, respectively) designed to exhibit these properties and to be utilized for the methods being disclosed. Pretreating the bioprosthetic heart valve biomaterial/s with one or more of these compounds as a step in the BHV manufacturing process prevents the deleterious effects of glycation per se and mitigates the deleterious effects of albumin/blood protein incorporation into the BHV by glycation cross-linking.

The present disclosure provides a method of protecting a bioprosthetic biomaterial from physiologic glycation and covalent incorporation of infiltrated circulating proteins, the method comprising the chemical modification of glycation-susceptible sites in bioprosthetic tissue to generate stable, permanent adducts that act as protecting groups against physiologic glycation, including but not limited to, treating the bioprosthetic biomaterial with a composition comprising a compound that; a) reacts directly or indirectly with glycation-susceptible sites in the bioprosthetic biomaterial to generate stable adducts that prevent the sites from reacting with physiologic glycation precursors; b) has similar reactivity with the glycation-susceptible sites as the physiologic glycation precursors; c) undergoes chemical rearrangements that allow for the resolution of intermediates to stable, permanent adducts that are unreactive or minimally reactive to glycation and do not follow glycation-like chemical rearrangements that result in the generation of glycation product-like moieties; and d) is minimally deleterious or non-deleterious to the function of the bioprosthetic biomaterial, in an amount sufficient to prevent or reduce physiologic glycation of the bioprosthetic biomaterial. In some embodiments the bioprosthetic biomaterial is intended for clinical implantation in a human. In certain embodiments the bioprosthetic biomaterial is a bioprosthetic heart valve, patch, urethral patch, aortic homografts, valve prostheses, stented valve, stentless valve, graft, dural graft, pulmonary ligament, artificial heart, or conduit. In particular embodiments the bioprosthetic biomaterial is a bioprosthetic heart valve. In some embodiments the bioprosthetic heart valve is located in a human, while in other embodiments the bioprosthetic heart valve is treated prior to implantation in a human. In various embodiments the composition comprises:

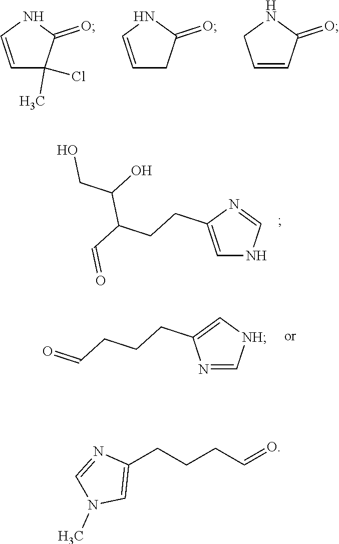

In certain embodiments the treatment renders the tissue resistant to the formation of heterocyclic and/or cross-linking glycation products without significantly diminishing formation of linear-chain and/or cell-signaling glycation products or vice-versa. In some embodiments the resultant tissue is utilized as a platform for decoupling the biological effects of the heterocyclic and/or cross-linking glycation products from those of linear-chain and/or cell-signaling glycation products or vice-versa. In other embodiments the resultant tissue is utilized as an experimental or laboratory implement for decoupling the biological effects of the heterocyclic and/or cross-linking glycation products from those of linear-chain and/or cell-signaling glycation products or vice-versa, including but not limited to cell culture incubations and chemical or biochemical analyses. In further embodiments the resultant tissue is utilized as a platform for the screening of therapeutic or drug compounds or interventional treatments, including but not limited to anti-glycation interventions.

The present disclosure also provides a method of protecting a bioprosthetic heart valve from structural valve degeneration, the method comprising treating the bioprosthetic heart valve or bioprosthetic heart valve tissue with a composition comprising:

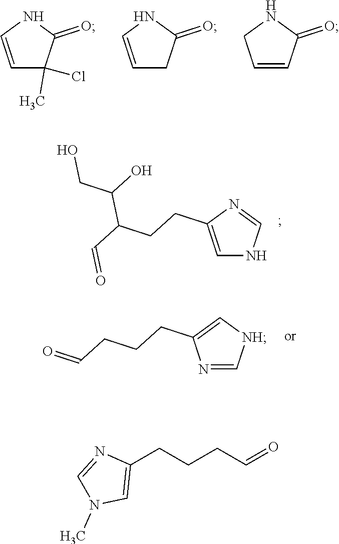

in an amount sufficient to prevent or reduce physiological glycation of the bioprosthetic heart valve. In certain embodiments the bioprosthetic heart valve is located in a human, while in some embodiments the bioprosthetic heart valve is treated prior to implantation in a human.

The present disclosure further provides a method of modifying a bioprosthetic biomaterial, the method comprising contacting the bioprosthetic biomaterial with a composition comprising:

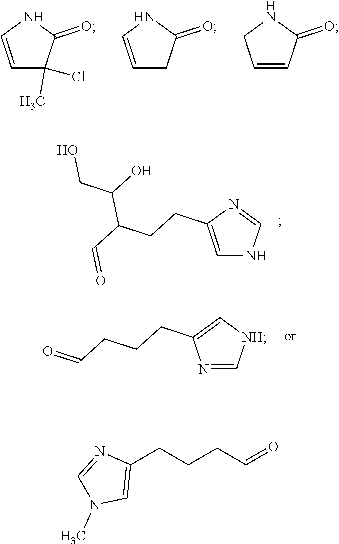

in an amount sufficient to prevent or reduce physiological glycation of the bioprosthetic biomaterial. In certain embodiments the bioprosthetic biomaterial is a bioprosthetic heart valve, patch or conduit. In particular embodiments the bioprosthetic biomaterial is a bioprosthetic heart valve. In certain embodiments the bioprosthetic heart valve is located in a human. In other embodiments the bioprosthetic heart valve is modified prior to implantation in a human.

The present disclosure additionally provides a method of treating a human having a bioprosthetic heart valve to prevent or reduce physiological glycation of the bioprosthetic heart valve, the method comprising administering to the human a therapeutically effective amount of a pharmaceutical composition comprising

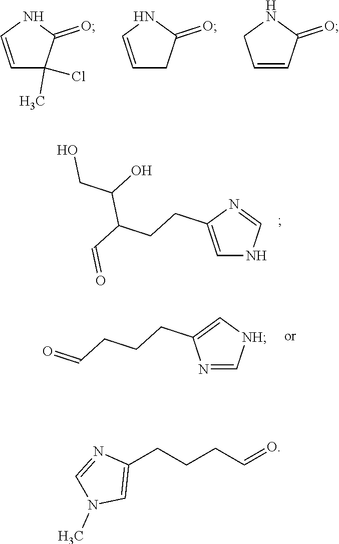

The present disclosure also provides a bioprosthetic biomaterial treated with a composition comprising a compound that: a) reacts directly or indirectly with glycation-susceptible sites in the bioprosthetic biomaterial to generate stable adducts that prevent the sites from reacting with physiologic glycation precursors; b) has similar reactivity with the glycation-susceptible sites as the physiologic glycation precursors; c) undergoes chemical rearrangements that allow for the resolution of intermediates to stable, permanent adducts that are unreactive or minimally reactive to glycation and do not follow glycation-like chemical rearrangements that result in the generation of glycation product-like moieties; and d) is minimally deleterious or non-deleterious to the function of the bioprosthetic biomaterial, in an amount sufficient to prevent or reduce physiologic glycation of the bioprosthetic biomaterial. In certain embodiments the bioprosthetic biomaterial is a bioprosthetic heart valve, patch, urethral patch, aortic homografts, valve prostheses, stented valve, stentless valve, graft, dural graft, pulmonary ligament, artificial heart, or conduit. In particular embodiments the bioprosthetic biomaterial is a bioprosthetic heart valve. In some embodiments the composition comprises:

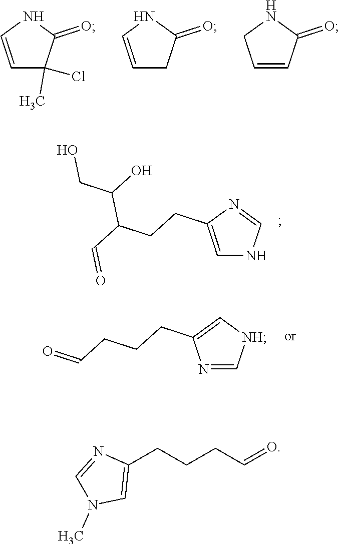

The present disclosure also provides methods of protecting a bioprosthetic biomaterial from physiologic glycation and/or covalent incorporation of infiltrated circulating proteins and/or calcification, the method comprising extended incubation of the bioprosthetic biomaterial in water or compatible buffers, such as phosphate-buffered saline, with or without stabilizers of carbonyl crosslinking, at elevated temperatures including human physiologic temperature (37° C.).

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. A simplified schematic of bioprosthetic tissue, exemplified by glutaraldehyde-fixed bovine pericardium. Glutaraldehyde crosslinks that impart tissue durability are assumed and not shown (though it is notable that these crosslinks occupy a significant proportion of tissue lysines).

FIG. 2. A simplified schematic of bioprosthetic tissue alteration during implantation.

FIG. 3. A simplified schematic of preservation of bioprosthetic tissue from physiologic glycation by the presently described strategy.

FIG. 4. The mechanism and major products of tissue glycation-susceptible site modification with 1,3-dihydro-2H-pyrrol-2-one. Mechanism 1 indicates Schiff base formation. Mechanism 2 indicates resolution of the Schiff base to a stable adduct.

FIG. 5. The conceptual mechanism and major products with 4-(1H-Imidazol-4-yl)butanal. Mechanism 1 indicates Schiff base formation. Mechanism 2 indicates resolution of the Schiff base to a stable adduct.

FIG. 6. Mitigation of Maillard browning due to in vitro glycation by anti-glycation tissue pre-treatment.

FIG. 7. Second harmonic generation imaging of collagen fiber bundles, showing apparent protection against collagen network disalignment caused by in vitro glycation with concomitant serum albumin infiltration by anti-glycation tissue pre-treatment.

FIG. 8. Immunohistochemistry using a standard anti-CML antibody to probe for the best-studied glycation product, N-carboxymethyl lysine, after control or anti-glycated glutaraldehyde-fixed bovine pericardium tissue discs were glycated under aggressive conditions known to efficiently generate CML (one week incubation at 37° C. in 50 mM glyoxal). Brown staining indicates presence of CML.

FIG. 9. Bar graph of the quantitation of uptake by glutaraldehyde-fixed bovine pericardium (Glut-BP) of radioactively-labeled glycation precursor after 24 hours of incubation at 37° C. in 50 mM ¹⁴C-radiolabeled glyoxal. Glut-BP was either kept in phosphate-buffered saline solution (PBS) in storage at 4° C. or placed in pre-incubation at 37° C. in PBS, PBS+1×1,5-dihydro-2H-pyrrol-2-one at 7.2 μmol/mg glut-BP, or PBS+1×4-(1-methyl-1H-imidazol-4-yl)butanal at 7.2 μmol/mg glut-BP.

### DETAILED DESCRIPTION OF THE INVENTION

The present disclosure addresses the discovery that the accumulation of glycation products, including advanced glycation end products (AGEs) and concomitant incorporation of infiltrated blood proteins in BHV leaflets, contributes to BHV SVD.

Heart valve disease at this time can only be treated surgically, with either valve replacement or repair. Bioprosthetic heart valves (BHV), fabricated from glutaraldehyde fixed heterografts, such as bovine pericardium (BP) or porcine aortic valves (PAV), are widely used in both cardiac surgery and in transcatheter valve replacements. Despite outstanding short term outcomes, BHV dysfunction due to structural valve leaflet degeneration (SVD) develops over time, frequently necessitating device replacement. Calcification is observed in the majority of SVD cases; however, 25% or more SVD cases are not associated with calcification. The inventors have demonstrated that BHV are susceptible to degenerative glycation. The inventors have also observed in BHV explant samples the presence of advanced glycation end products (AGE), including carboxymethyl-lysine (CML) a ligand for the receptor for AGE (RAGE) and glucosepane, the most abundant AGE crosslink.

Based on research on the degeneration of clinically-implanted bioprosthetic heart valves, the inventors have also determined that infiltration and incorporation of serum albumin into the valve tissue occurs in clinical valves, modifies tissue properties/valve performance, and contributes to valve degeneration. Albumin is by far the most abundant protein in the blood-accounting for over half of all blood proteins by mass; therefore, it is inherently uniquely important with regard to blood components in this respect. Bioprosthetic tissues from which these valves are manufactured have no resistance to the infiltration of albumin from surrounding fluid; as a result, albumin infiltrates into the tissue immediately upon exposure to a fluid containing it (i.e., blood, in the clinical context) and throughout the depth of the tissue, both in vitro and in vivo. Albumin can be permanently incorporated into the valve tissue by glycation via physiologically-relevant precursors, such as sugars and sugar-derived dialdehydes. In this context, albumin incorporation has both glycation-related and glycation-independent effects on valve tissue and performance.

Based on these findings, the present disclosure provides compositions and methods for improved performance and durability testing of BHV, as well as anti-glycation compositions for the protection of BHV from physiological glycation.

Additional AGE/RAGE Therapeutics

The anti-glycation agents described herein can also be used with one or more AGE/RAGE therapeutic agents, including, but not limited to, anti-oxidants, pyridoxamine (Pereira-Simon et al., PLoS One 11:e0159666, 2016; Brodeur et al., PLoS One 9:e85922, 2014), AGE breakers that disrupt AGE structure (Brodeur et al., PLoS One 9:e85922, 2014, Candido et al., Circ. Res. 92:785-792, 2003) and RAGE-specific receptor antagonists (Cai et al., Cell. Mol. Neurobiol. 36:483-495, 2016; Deane et al., J. Clin. Invest. 122:1377-1392, 2012). Additional agents for use with the presently disclosed anti-glycation agents include, but are not limited to, PHOTOFIX® (CryoLife, Inc. Kennesaw, Ga.), pentagalloyl glucose, XLF-III-43 (U.S. Pat. No. 8,729,280), irbesartan, TM2002 (Izuhara et al., Nephrol. Dial. Transplant. 23:497-509, 2007), diclofenac, pioglitazone, metformin, pentoxifylline and N-phenacylthiazolium bromide, as well as compounds disclosed in U.S. Patent Application Publication Number 2014/0127804, International Patent Application Publication Number WO 2013/032969, and U.S. Pat. Nos. 6,093,530, 6,552,077, 10,016,450.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain an effective amount of an anti-glycation agent described herein, which can be in purified form, together with a suitable amount of carrier so as to provide the form for proper administration to the subject.

The term “formulation” refers to preparing an agent in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating structural valve degeneration in a subject in need thereof comprising administration of a therapeutically effective amount of an anti-glycation agent described herein.

Methods described herein are generally performed on a subject in need thereof. A subject in need of the therapeutic methods described herein can be a subject having, diagnosed with, suspected of having, or at risk for developing structural valve degeneration. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of an anti-glycation agent described herein is, for example, that amount that would cause the desired therapeutic effect in a subject while minimizing undesired side effects. In various embodiments, an effective amount of an agent described herein can substantially inhibit structural valve degeneration, ameliorate structural valve degeneration, slow the progress of structural valve degeneration, or limit the development of structural valve degeneration.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an anti-glycation agent described herein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat or prevent structural valve degeneration.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an anti-glycation agent described herein can occur as a single event or over a time course of treatment. For example, an anti-glycation agent described herein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for structural valve degeneration.

An anti-glycation agent described herein can be administered simultaneously or sequentially with another agent, such as an antibiotic, an anti-inflammatory, or another agent. For example, an anti-glycation agent described herein can be administered simultaneously with another agent, such as an antibiotic or an anti-inflammatory. Simultaneous administration can occur through administration of separate compositions, each containing one or more of an anti-glycation agent described herein, an antibiotic, an anti-inflammatory, or another agent. Simultaneous administration can occur through administration of one composition containing two or more of an anti-glycation agent described herein, an antibiotic, an anti-inflammatory, or another agent. An anti-glycation agent described herein can be administered sequentially with an antibiotic, an anti-inflammatory, or another agent. For example, an anti-glycation agent described herein can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the agent of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for agent delivery can: provide for intracellular delivery; tailor agent release rates; increase the proportion of agent that reaches its site of action; improve the transport of the drug to its site of action; allow co-localized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration or testing. Such kits can facilitate performance of the methods described herein. When supplied as a kit, the different components of the composition can be packaged in separate containers and admixed immediately before use. Components include, but are not limited to the anti-glycation agents described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Anti-Glycation Compounds

Prosthetic valves address Heart Valve Diseases (HVD), mostly related to stenosis, insufficiency, or endocarditis. HVD increasingly diminish quality of life throughout their progression, eventually leading to heart failure and death if left untreated. For examples, severe aortic valve stenosis (AS) exhibits higher two-year higher mortality rates than most cancers if not addressed by aortic valve replacement (AVR). When any of the four heart valves is replaced, a prosthetic valve is surgically implanted. Heart Valve Disease (HVD) and associated comorbidities are a major clinical and socioeconomical burden in the US and worldwide. Both the number of procedures and the costs of intervention are expected to rise exponentially in coming years, with a projected number of over 300,000 procedures each year and a market approaching $9 billion by 2050 in the US alone. Patients of all ages are impacted. Developing countries are particularly affected and underserved by interventional cardiologists and surgical care. In low income countries—where less than 0.5% of patients have access to surgical units—it has been estimated that 35 to 70 million patients are affected. Importantly, patients often experience symptoms, recurrence, and failure of prosthetic valves even after valve repair/replacement, and no mitigating therapies before or after valve replacement exist.

The two main categories of prosthetic heart valve are bioprosthetic heart valves (BHV) and mechanical heart valves. BHV are increasingly favored over mechanical valves, which are less versatile and generally require lifelong anti-coagulation therapy. Additionally, BHV are the only option for more recently-developed non-surgical, transcatheter-based valve replacements, which are widely expected to be the standard of care for most aortic valve replacements within ten years. Currently, bioprosthetic valves account for roughly 80% of prosthetic heart valve implantations. Transcatheter or percutaneous heart valve replacements, such as transcatheter aortic valve replacements (TAVR), constitute a more recently-developed class of bioprosthetic valves that enable heart valve replacement without surgery. TAVR expands the patient population available for bioprosthetic valve implantation and is quickly becoming the standard of care for aortic valve disease due highly-reduced invasiveness of the procedure, reduced number of hospital days, and faster recovery. However, BHV, being modified biological material, are limited by biochemically-driven degeneration of the structural protein molecules of the leaflet tissue, termed structural valve degeneration (SVD), which involves biochemical and structural modification of valve leaflet tissue matrix and resultant degeneration of functionality. These mechanism(s) fundamentally limit the lifespan of BHV, currently to an overall average between 10 and 15 years. There are also indications that SVD is even more limiting in transcatheter valves, as the crimping necessary for catheter-based, minimally-invasive delivery may exacerbate structural defects. SVD and BHV failure requires implantation of another BHV, via either surgery or percutaneous delivery, with the associated costs, risks, and quality of life disruptions.

Transcatheter techniques can spare the patient repeated open-heart surgery in eligible first-time AVR cases and where the emerging valve-in-valve procedure is applicable; however, in most BHV failure cases, invasive reoperation is required, presenting significant physical trauma and associated risks, cost, and quality of life diminishment.

The inventors have identified that physiological glycation, a spontaneous chemical process by which sugars and sugar-derived molecules form adducts on proteins and cross-link proteins to each other, and infiltration and incorporation into BHV tissue of blood proteins—most prominently serum albumin—represent synergistic mechanisms broadly underlying SVD and BHV failure.

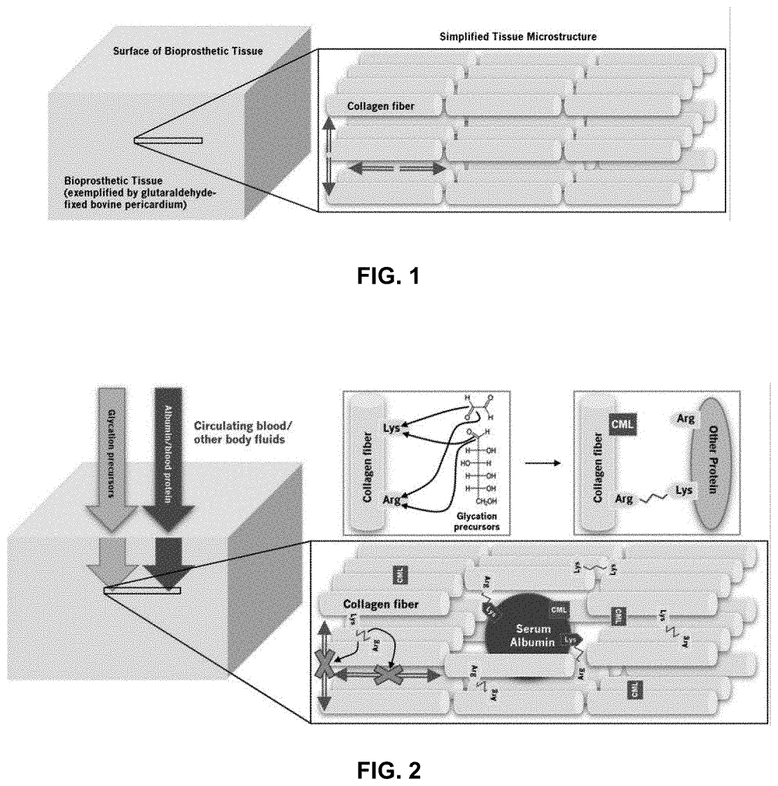

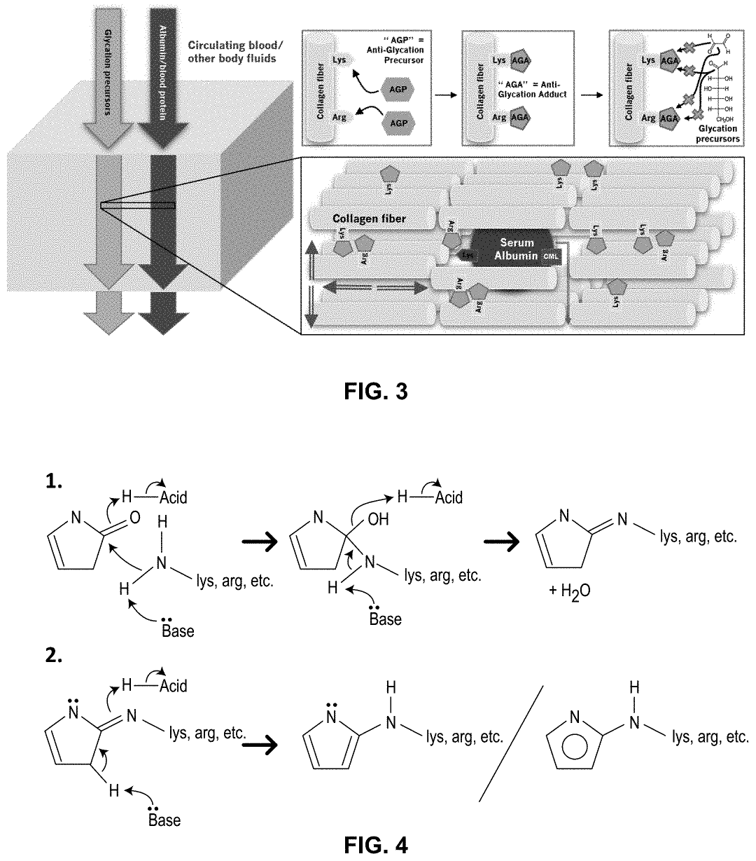

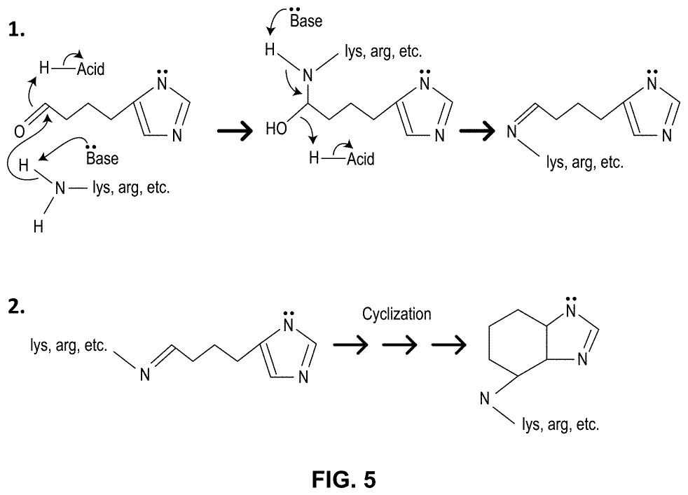

The base tissue structure of bioprosthetic tissue is shown in FIG. 1. These tissue are always mostly composed of collagen type I and often are up to ˜90% collagen, for which functionally-significant glycation susceptibility is well-characterized. Thus, only collagen is shown for simplicity. Collagen is arranged in partially-aligned stacks. The fibers dissipate force during tissue biomechanical activity by sliding past each other and by stacks compressing and relaxing in the Z dimension, indicated by arrows.

As detailed above, the inventors have identified that physiological glycation, a spontaneous chemical process by which sugars and sugar-derived molecules form adducts on proteins and cross-link proteins to each other, and infiltration and incorporation into BHV tissue of blood proteins—most prominently serum albumin—represent synergistic mechanisms broadly underlying SVD and BHV failure (FIG. 2). Implantation of such tissues initiates surface exposure to blood and/or body fluids. Small molecule glycation precursors and blood proteins in various states of glycation freely infiltrate into these tissues from the blood. Glycation precursors and intermediately-glycated proteins react with glycation-susceptible sites, generating inflammatory-stimulating adducts (e.g., CML), collagen-collagen crosslinks, and collagen-infiltrated protein crosslinks. Glycation crosslinking permanently incorporates infiltrated proteins, which, in turn, can mediate longer-distance crosslinking between collagen fibers in different layers, exacerbating extracellular matrix disruption caused by both glycation and protein infiltration. Glycation crosslinking interferes with tissue dissipation of force, indicated by X's in FIG. 2.

Glycation occurs spontaneously and non-enzymatically when sugar-derived aldehydes condense with protein amine and guanidinium groups. Advanced glycation end products (AGEs) are biochemically irreversible chemical endpoints of the glycation process. AGEs can be simple protein adducts, derivative small molecules (e.g., acrylamide), or crosslinks. AGEs exert a variety of biological effects via two main pathways: 1) ECM degeneration; and 2) modulation of cell phenotypes via receptor signaling. AGEs are associated with tissue degeneration in a variety of diseases. N-Carboxymethyllysine (CML) is the best-studied signaling AGE, and glucosepane is the most physiologically abundant crosslinking AGE.

Endogenous formation of AGEs has been described by three different paths in vivo: the non-enzymatic Maillard reaction; the polyol-pathway; and lipid peroxidation. During all three reactions, formation of AGEs occurs over formation of reactive carbonyl compounds, such as glyoxal, methylglyoxal and 3-deoxyglucoson. If detoxification is impaired they are able to react further until the formation of irreversible AGEs. Sugars and sugar-derived dialdehydes are representatives of the general class of glycation precursor chemicals. Notably, they all include nitrogen-reactive aldehydes or ketones, which are nucleophilically attacked by side-chain nitrogens of amino acids, primarily lysine, arginine, histidine and tryptophan. These are the reactive components. The adjacent —OH groups or second aldehydes that can become —OH groups are important for Amadori rearrangement, which is an avenue for the Schiff base to be resolved that leads to glycation end products.

AGEs modify and degenerate collage network biomechanical properties. Collagen is the majority (˜90% by mass) component of bioprosthetic heart valve biomaterials. This effect occurs largely due to inter- and intramolecular crosslinking, which disrupts collagen network dissipation of force.

The inventors have developed both a strategy to protect the major susceptible chemical sites of bioprosthetic valve tissue from physiological glycation and small molecule compounds designed to achieve this purpose. These sites are nitrogenous chemical groups in tissue protein amino acid (primarily lysine and arginine) side chains that are prone to spontaneous reaction with sugar-derived aldehyde and ketone groups, which is the initiating step of glycation (FIG. 2). The presently described anti-glycation tissue pretreatment generates protective adducts at potential glycation sites that block glycation precursors from reacting with and glycating these sites. Additionally, this blocks the permanent incorporation (via glycation crosslinking) of infiltrated proteins by glycation cross-linking, whether the mechanism be de novo glycation events between infiltrated protein and tissue proteins or the condensation of intermediately-glycated blood protein (exemplified by Amadori-album in) capable of formation of glycation crosslinks without any additional small molecule glycation precursor. Blood proteins may still freely infiltrate and diffuse out, as indicated, but will cause less disruption to the tissue structure and will not be permanently incorporated.

The subject compounds are designed to have similar reactivity with these sites as glycation precursors, and the ability to resolve the Schiff base to a stable end product, but avoid Amadori rearrangement and go in a direction that does not lead to advanced glycation end products, but instead preserves native side chain chemistry and sterics. By pretreating the bioprosthetic heart valve biomaterial with one or more of these compounds as a step in the BHV manufacturing process, they chemically react with these sites to generate stable protecting groups that prevent glycation from occurring after implantation. This prevents the deleterious effects of glycation per se and mitigate the deleterious effects of albumin/blood protein incorporation into the BHV by glycation cross-linking. The compounds are designed to generate defined chemical adducts that are non-deleterious to protein function, being relatively small in size, bearing no capability for cross-linking, bearing relatively similar chemical properties to the natural side chains being modified, and/or coordinating water molecules naturally resident in collagen fibril void spaces to mask any side chain polarity and molecular shape differences.

Two specific compound families are disclosed herein for this purpose. Class 1 constitutes a family of molecules with a scaffold consisting of 2-Pyrrolidinone with a single ring double bond. Potentially useful double bonds can exist at either the 3-4 position—giving rise to 1,3-Dihydro-2H-pyrrol-2-one—or the 2-3 position—giving rise to 1,5-Dihydro-2H-pyrrol-2-one. The basis of the design for this scaffold is as follows. The carbonyl provides a reactive group similar to sugar-derived carbonyls for reaction with protein side chains. Upon reaction, a Schiff base will be formed on the previously carbonyl carbon, as in glycation. Spontaneous imine-enamine tautomerism that naturally follows Schiff base formation will cause migration of the previously-carbonyl double bond into the ring structure. The existence of a previous double bond at either of these positions in the ring will allow for a stable resolution of the Schiff base by generating pyrrole aromaticity with the enamine double bond and the ring nitrogen lone pair, thus stabilizing the enamine form and prevent backsliding to the Schiff base. The end product of glycation-protective reaction by Class 1 compounds will be a pyrrole side chain adduct to the reacting nitrogen, to which it will be connected by a stable amine-like bond. A pyrrole adduct is chemically similar to an imidazole adduct, the side chain group of histidine, which is the most conservative amino acid substitution for lysine and arginine. It contains an aromatic ring nitrogen, is not bulky compared to most AGE substitutions, and is chemically stable and not amenable to crosslinking.

The double-bond positioning in 1,5-Dihydro-2H-pyrrol-2-one requires it to shift in order to generate aromaticity upon imine-enamine tautomerization, which requires the shuffling of protons and electrons and likely requires basic conditions. The double-bond positioning in 1,3-Dihydro-2H-pyrrol-2-one requires no such molecular rearrangements and can readily accommodate the imine-enamine tautomerization to generate pyrrole aromaticity. Therefore, the 1,3-Dihydro-2H-pyrrol-2-one scaffold is preferred in certain embodiments. However, 1,3-Dihydro-2H-pyrrol-2-one requires custom chemical synthesis, while 1,5-Dihydro-2H-pyrrol-2-one is commercially available. Using 1,5-Dihydro-2H-pyrrol-2-one, data have been generated indicating a dramatic protection of anti-glycation pre-treated glutaraldehyde-fixed bovine pericardial discs against optical browning—a classic hallmark of glycation—under very aggressive glycation conditions (50 mM glyoxal, 50 mM methylglyoxal, or 50 mM glyoxal+5% HSA for 7 days at 37° C.). However, specific immunohistochemical analysis using antibodies to probe CML formation in response to glyoxal and CEL in response to methylglyoxal indicate that both are still formed similarly as in untreated tissue in anti-glycated tissue. Even better results in certain embodiments will occur with 1,3-Dihydro-2H-pyrrol-2-one.

Specific derivatives in this class are described below. Namely, a chlorine atom and/or a methyl group can be used in place of hydrogens to decorate ring position 3. The chlorine atoms can serve as an electron-withdrawing group for the ketone, rendering it more reactive to nucleophilic attack by the lone pairs of protein side chain amine and guanidinium nitrogens. The chlorine atom then functions as a leaving group, facilitating the enamine tautomer of the imine-enamine tautomerism that naturally follows Schiff base formation. The methyl group blocks side reactions at this key carbon, stabilizes the carbocation intermediate and facilitates chloride ion dissociation during double bond migration, and discourages ancillary SN2-like reactions by stock compound with itself.

Class 2 is based on a natural chemical reaction previously observed (Vasquez et al., Amino Acids 3:81-94, 1992). This reaction indicated spontaneous and fast formation of a 6-member heterocycle subsequent to Schiff base formation between histidine and pyridoxal-5′-phosphate via reducibility of the sterically well-positioned histidine carbon-carbon double bond. The Class 2 scaffold, 4-(1H-Imidazol-4-yl)butanal, was designed to impose this reaction on glycation-susceptible amino acid sites. Thus, the scaffold is an imidazole modified with a functionalized 4-carbon chain on one of the chemically equivalent imidazole carbons that shares a double bond with another carbon. The final carbon in the chain bears an aldehyde to enable reaction similar to glycation precursors with the target sites. Subsequent to Schiff base formation the Schiff base can react with the imidazole carbon-carbon double bond, forming a new, 6-carbon cycle via a new bond between the Schiff base carbon and the imidazole double-bond carbon opposite to the carbon bearing the chain. This allows for stable resolution of the Schiff base, leaving a stable secondary amine-like bond to the original amino acid side chain.

Specific derivatives in this class are described below. The carbon alpha to the carbonyl (aldehyde) can be decorated by carbon-carbon bond to a water-sequestering moiety such as ethylene glycol, which sequesters local water molecules and generates a solvation shell around the Schiff base formed at the former carbonyl subsequent to its formation. This protects the Schiff base from hydrolytic cleavage and reversal of the initial reaction. The water-sequestering moiety need not necessarily be ethylene glycol specifically and alternative moieties of similar functionality, including, but not limited to, polyethylene glycol and related moieties can be used in certain embodiments. Similarly, chemically inconsequential decorations that may facilitate chemical synthesis or purification, such as methyl groups on the imidazole ring, can be used in certain embodiments.

Compound 1 is a Class I molecule, a halogen-modified 5-membered heterocycle with a reactive carbonyl (ketone).

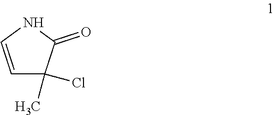

The ring contains one nitrogen atom, adjacent to the carbonyl carbon. A chlorine atom and a methyl group modify the carbon on the opposite side of the carbonyl from the nitrogen. A double bond lies meta to the carbonyl, joining the two unmodified carbons. The ketone provides a reactive group similar to sugar-derived carbonyls for reaction with protein side chains. The chlorine atoms serves as an electron-withdrawing group for the ketone, rendering it more reactive to nucleophilic attack by the lone pairs of protein side chain amine and guanidinium nitrogens. Upon reaction, a Schiff base will be formed on the previously carbonyl carbon, as in glycation. The chlorine atom will then function as a leaving group, facilitating the enamine tautomer of the imine-enamine tautomerism that naturally follows Schiff base formation. This will favor migration of the double bond from the Schiff base to the halogenated ring carbon. This double bond migration into the ring, in turn, will generate aromaticity with the ring nitrogen lone pair and the double bond meta to the former carbonyl carbon, strongly stabilizing this positioning of the former Schiff base double bond. The optional methyl group is intended to block side reactions at this key carbon, stabilize the carbocation intermediate and facilitate chloride ion dissociation during double bond migration, and discourage ancillary SN2-like reactions by stock compound. The end product of glycation-protective reaction by Compound 1 will be a pyrrole side chain adduct to the reacting nitrogen, to which it will be connected by a stable amine-like bond. Pyrrole is chemically highly similar to imidazole, the side chain group of histidine, which is the most conservative amino acid substitution for lysine and arginine. It contains an aromatic ring nitrogen, is not bulky compared to most AGE substitutions, and is chemically stable and not amenable to crosslinking. The pyrrole-precursor ring can also be derivatized with ethylene glycol/PEG side chain moiety to sequester local water molecules to mask the side chain and protect the Schiff base from hydrolytic cleavage. Compound 1 is a 1,3-dihydro-2H-pyrrol-2-one derivative. A facile organic synthesis protocol for such compounds is known in the art.

Compound 2 is a related Class I molecule, 1,3-dihydro-2H-pyrrol-2-one

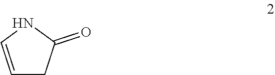

Known to chemistry as a class of compound that is useful for reactions with nucleophilic nitrogens (which is what it is intended to do). This compound class has not been used to block glycation or on tissue bioprosthetic materials, and facile synthesis of compounds in this family has been described (Wang et al., Tetrahedron Lett. 58:847-850, 2017).

The mechanism and major products of compound 2 are shown in FIG. 4. Straightforward mechanism utilizes a backbone that allows for a non-Amadori avenue for essentially one-step resolution of the Schiff base to a stable product. Schiff bases inherently undergo imine-enamine tautomerism, which involves the shifting of the double bond away from the imine to the adjacent carbon. This precursor traps the Schiff base double bond in stable pyrrole aromaticity when this tautomerism occurs, preventing backsliding and generating a stable product. Pyrrole groups are ubiquitous in natural biochemistry; thus, these adducts are expected to be both stable and minimally disruptive, as they are already encountered by other tissue biochemicals, are sterically small, and preserve the nitrogenous and polar natures of glycation-susceptible amino acid groups. In contrast, glycation products such as CML replace a positive charge with a negative charge.

Compound 3 is a related Class I molecule, 1,5-dihydro-2H-pyrrol-2-one:

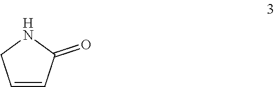

Known to chemistry as a class of compound that is useful for reactions with nucleophilic nitrogens (which is what it is intended to do). This compound is readily available from Aldrich. Ring double bond placement enables competitive 1-4 additions that reduce anti-glycation efficacy relative to the 1, 3 isomer.

Compound 4 is a Class 2 molecule, an imidazole modified with a functionalized 4-carbon chain on one of the chemically equivalent carbons that shares a double bond with another carbon.

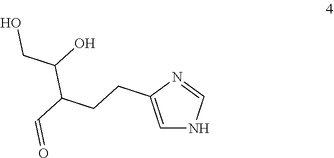 [SMILES: O=CC(CCc1c[nH]cn1)C(O)CO]

The final carbon in the chain bears an aldehyde to enable reaction similar to glycation precursors with the target sites. The carbon alpha to the carbonyl (aldehyde) is directly linked by carbon-carbon bond to an ethylene glycol moiety, which is intended to sequester local water molecules and generate a solvation shell around the Schiff base formed at the former carbonyl subsequent to its formation. This should protect the Schiff base from hydrolytic cleavage and reversal of the initial reaction. Subsequent to Schiff base formation and enabled by its protection by the water-sequestering moiety, which can be ethylene glycol (polyethylene glycol or related moieties offer additional options), the Schiff base can react with the imidazole carbon-carbon double bond, forming a new, 6-carbon cycle via a new bond between the Schiff base carbon and the imidazole double-bond carbon opposite to the carbon bearing the chain. This allows for stable resolution of the Schiff base, leaving a stable secondary amine-like bond to the original amino acid side chain.

Compound 5 is a related Class 2 molecule, 4-(1H-Imidazol-4-yl)butanal.

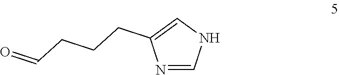

This compound was designed based on the spontaneous, non-enzymatic Schiff base formation and stable resolution via an adjacent imidazole group as previously described (Vasquez et al., Amino Acids 3:81-94, 1992). The mechanism and major products of this compound is shown in FIG. 14.

Compound 6 is a related Class 2 molecule, 4-(1-methyl-1H-imidazol-4-yl)butanal:

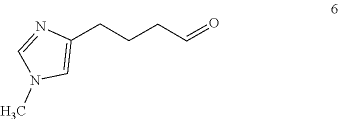

These compounds were designed ab initio with certain principles in mind: 1) Mimicking reactivity of physiological glycation precursors in general with glycation sites (nucleophilic nitrogens of the side chains of amino acids like lysine and arginine) in bioprosthetic tissues. The carbonyls are the glycation-mimic reactive centers, designed to form Schiff bases with glycatable protein side chain nitrogens; 2) Following a chemical path other than Amadori rearrangement to resolve the Schiff bases formed upon reaction and generate stable adducts; and 3) The stable adducts generated should be non-deleterious/minimally modify the sterics, chemical properties, and interactions of the modified side chains, and should render the reaction sites inert/resistant to physiologic glycation.

A general method flow chart is described below. First, standard 0.6% glutaraldehyde fixation protocol (+/−any additional technology, e.g., anti-calcification). Second, wash protocol in phosphate-buffered saline to remove residual unreacted glutaraldehyde or other compounds. Third, incubation in subject compounds dissolved in, for example, phosphate-buffered saline at 37° C. for 6 weeks. Fourth, wash protocol in phosphate-buffered saline to remove residual unreacted anti-glycation precursor. Fifth, storage (usually in 0.2% glutaraldehyde.

Anti-glycation pretreatments have been performed for 1-, 2-, 4-, and 6-week-long durations for both 1,5-dihydro-2H-pyrrol-2-one as well as 4-(1-methyl-1H-imidazol-4-yl)butanal, and diversified 1-week glycation reactions have been applied to these samples. Namely, these conditions are 50 mM glyoxal, 50 mM methylglyoxal, and 50 mM glyoxal+5% human serum albumin. An antibody for N-carboxyethyl lysine, a major product of methylglyoxal glycation, was utilized for immunohistochemical staining.

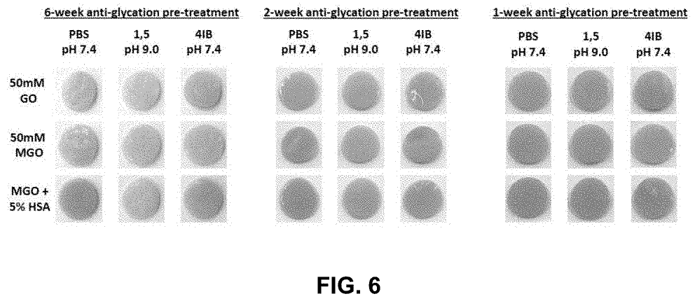

“Maillard browning” is a classic and straightforward visual indicator of glycation. Formation of glycation products causes brown hues to develop in glycated tissues and proteinaceous materials. As a preliminary endpoint for initial anti-glycation efficacy studies, the inventors sought to evaluate prevention of browning due to glycation in glutaraldehyde-fixed bovine pericardium, the most common bioprosthetic heart valve biomaterial (and also widely used in other bioprosthetic implants), by pretreatment with the designed anti-glycation compounds. Short term (3-day) and long-term (6-week) protective incubation durations at physiological temperature (37° C.) were chosen as a starting point. Anti-glycation was followed by extensive washing of tissue in phosphate-buffered saline (PBS) to remove any unreacted anti-glycation precursor and extreme glycation conditions (1-week incubation in 50 mM doses of either methylglyoxal or glyoxal, two of the most reactive physiologically-relevant glycation precursors, or 50 mM glyoxal+5% human serum albumin, which exacerbates browning). Treatments and evaluation of browning have been performed with 1,5-dihydro-2H-pyrrol-2-one (“1,5,” compound 3) and 4-(1H-Imidazol-4-yl)butanal (“4IB”). The results are shown in FIG. 6.

Results provide several notable and informative observations. There is dramatic diminishment of tissue browning due to aggressive glycation with either glycation precursor and precursor plus albumin after 6 weeks of anti-glycation pretreatment with 1,5 applied at slightly basic pH 9.0. Protection from browning is less pronounced with 4IB; this may be due to the fact that the compound itself is brightly yellow-brown and pre-treatment itself changes the color of the tissue. Shorter anti-glycation pre-treatments demonstrate progressively diminished differences in hue between controls and anti-glycated discs, indicating that observed results at 6 weeks of pre-incubation are real and due to extent of anti-glycation precursor reaction with tissue. Additionally, color of tissue treated with 1,5 for 6 weeks was identical after exposure to glycation precursor alone or precursor plus albumin, indicating that 1,5 effectively blocks incorporation of glycated (browned) albumin as well as glycation-based browning of the tissue itself. However, the placement of the double bond in this isomer enables possible 1,4 addition reactions across the double bond and carbonyl that would compete with the intended Schiff base formation and tautomerization, which form the anti-glycation moieties, potentially reducing efficiency. The 1,3 isomer was is specifically designed to disallow such side reactions with respect to the intended chemistry. Similarly, 4IB lacks any moieties to stabilize Schiff base upon formation, potentially reducing anti-glycation efficiency. Thus, related compounds can increase anti-glycation efficacy.

Tissue browning results also demonstrate progressively-reduced browning of tissue under extreme glycation conditions by simple long-term incubation at 37° C. in PBS. Elevated-temperature incubation may induce the stabilization of residual, unreacted glutaraldehyde carbonyl groups with tissue nucleophilic groups at which glycation can occur, thus blocking these tissue sites against glycation and resolving glutaraldehyde-derived carbonyl groups known to contribute to calcification during clinical implantation. However, these modifications are likely to be labile, as it has been demonstrated that glutaraldehyde-derived crosslinks in glut-BP reverse over time due to implantation in animals, and incubation at 37° C. in PBS alone did not minimize tissue browning in the presence of albumin alongside glycation precursor. This suggests that it did not block glycation-based incorporation of soluble protein.

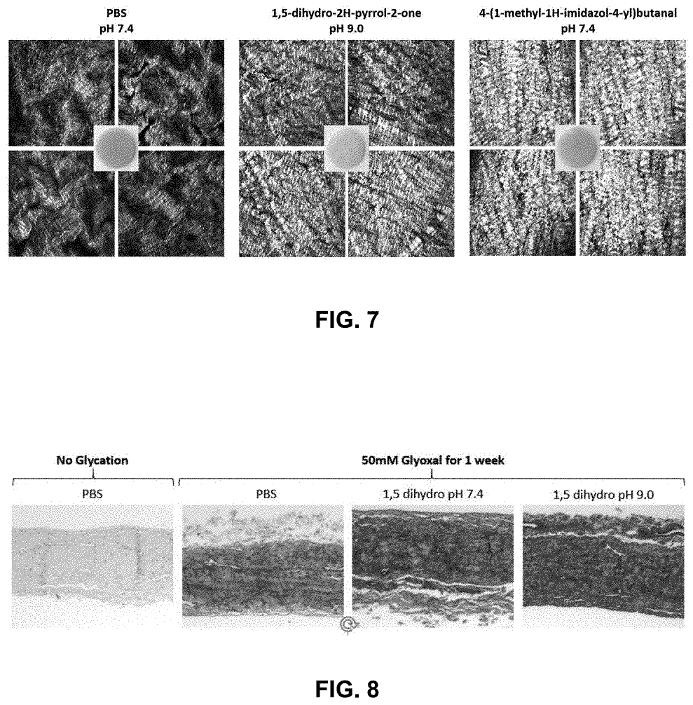

Second-harmonic generation imaging of collagen fiber bundle alignment was employed to assay protection against glycation- and albumin incorporation-based structural disruption of tissue collagen. Results are shown in FIG. 7. 6-week anti-glycated tissue discs were imaged following 1 week of glycation with 50 mM glyoxal in the presence of 5% human serum albumin. Untreated control discs indicate significant disalignment and convolution of collagen fiber bundles, whereas anti-glycated discs reveal highly-preserved alignment of fiber bundles, indicating that anti-glycation pre-treatment effectively blocked structurally-disruptive glycation product formation and incorporation of infiltrated albumin.

As a preliminary specific chemical analysis, immunohistochemistry was performed using a standard anti-CML (AbCAM #ab27684 @ 1:5000 dilution) antibody to probe for the best-studied glycation product, N-carboxymethyl lysine, after control or anti-glycated glutaraldehyde-fixed bovine pericardium tissue discs were glycated under aggressive conditions known to efficiently generate CML (one week incubation at 37° C. in 50 mM glyoxal). Brown staining indicates presence of CML. The results are shown in FIG. 8.

Immunohistochemistry results indicate that CML formation was not mitigated by pretreatment with 1,5-dihydro-2H-pyrrol-2-one for 6 weeks. Notable, CML is a non-pigmented, non-crosslinking linear adduct. Pigmented glycation products are generally heterocyclic and/or polymeric. Many heterocyclic glycation products are crosslinks, and most known crosslinks require both lysine and arginine from the protein. These results therefore indicate that 1,5-dihydro-2H-pyrrol-2-one and 4-(1-methyl-1H-imidazol-4-yl)butanal, under these reaction conditions, may preferentially block the formation of heterocyclic glycation products and glycation products that require multiple glycation precursor molecules and molecular rearrangements to form. Further, these results suggest that 1,5-dihydro-2H-pyrrol-2-one and 4-(1-methyl-1H-imidazol-4-yl)butanal, under these reaction conditions, react preferentially with arginines and/or other residues besides lysine that are required for pigmented glycation products to form. The specific glycation products assayed are purely lysine-directed glycation products. Mitigation of polymeric/heterocyclic and crosslinking glycation products constitutes a significant improvement over standard bioprosthetic tissue. Utilization of related compounds and treatment protocols within the scope of this tissue modification paradigm can mitigate linear-chain and cell signaling glycation products and lysine-directed glycation products and further reduce overall glycation as well.

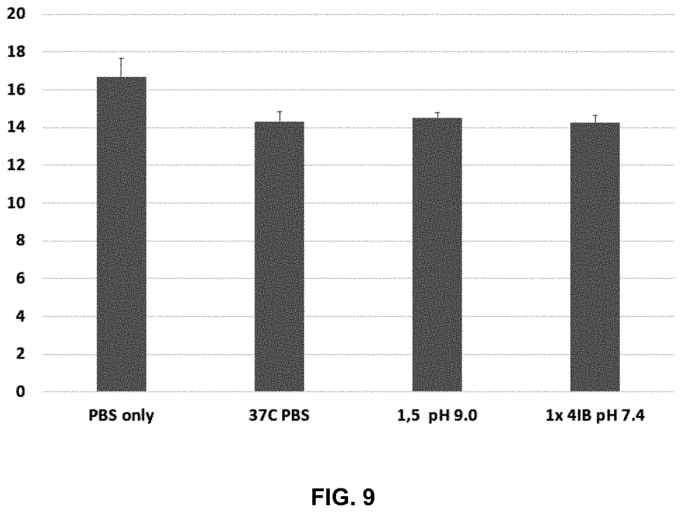

The above interpretations are supported by quantitation of uptake of glycation precursor by glut-BP using carbon-14-radiolabeled glyoxal, which indicates approximately 15% decreases in overall glycation versus by an extreme concentration (50 mM) of glyoxal in glut-BP pre-treated by 6-week incubation at 37° C. in PBS alone or 1×1,5 or 1×4IB, shown in FIG. 9. Thus, anti-glycation incubation of glut-BP under the tested conditions significantly reduces overall glycation while retaining the formation of non-pigmented glycation products.

Intact, clinical-grade bioprosthetic heart valves are subject to anti-glycation pre-treatments with the presently described compounds and assay any effects on baseline valve performance under physiologic conditions as well as any protective effects against the deterioration of valve performance caused by glycation and serum albumin incorporation. Specific biomechanical analyses, including mass spectrometry, fluorescamine-based assay of tissue free amines, and 9,10-Phenanthrenequinone-based assay of tissue free arginines, are performed to assay anti-glycation treatment effectiveness on preserving tissue biomechanical properties, such as stiffness and elasticity. Cell biological assays are used to assay anti-glycation treatment effects on cell adhesion and stimulation of inflammatory responses. Rat subcutaneous implantation is utilized to assay efficacy of anti-glycation pre-treatment in mitigating glycation, protein infiltration, and SVD in vivo.

Concepts singly employed in either compound—for example, a water sequestration moiety—can be cross-applied to generate derivatives of the other.

What is claimed is:

1. A method of protecting a bioprosthetic biomaterial from physiologic glycation and covalent incorporation of infiltrated circulating proteins, the method comprising treating the bioprosthetic biomaterial with a composition comprising a compound that:
a. reacts directly or indirectly with glycation-susceptible sites in the bioprosthetic biomaterial to generate stable adducts that prevent the sites from reacting with physiologic glycation precursors;
b. has similar reactivity with the glycation-susceptible sites as the physiologic glycation precursors;
c. undergoes chemical rearrangements that allow for the resolution of intermediates to stable, permanent adducts that are unreactive or minimally reactive to glycation and do not follow glycation-like chemical rearrangements that result in the generation of glycation product-like moieties; and
d. is minimally deleterious or non-deleterious to the function of the bioprosthetic biomaterial,
in an amount sufficient to prevent or reduce physiologic glycation of the bioprosthetic biomaterial.

2. The method of claim 1, wherein the bioprosthetic biomaterial is intended for clinical implantation in a human.

3. The method of claim 1, wherein the bioprosthetic biomaterial is a bioprosthetic heart valve, patch, urethral patch, aortic homografts, valve prostheses, stented valve, stentless valve, graft, dural graft, pulmonary ligament, artificial heart, or conduit.

4. The method of claim 3, wherein the bioprosthetic biomaterial is a bioprosthetic heart valve.

5. The method of claim 4, wherein the bioprosthetic heart valve is located in a human.

6. The method of claim 4, wherein the bioprosthetic heart valve is treated prior to implantation in a human.

7. The method of claim 1, wherein the composition comprises:

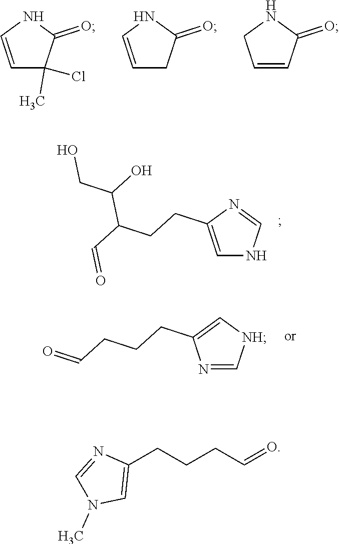

8. A method of protecting a bioprosthetic heart valve from structural valve degeneration, the method comprising treating the bioprosthetic heart valve with a composition comprising: 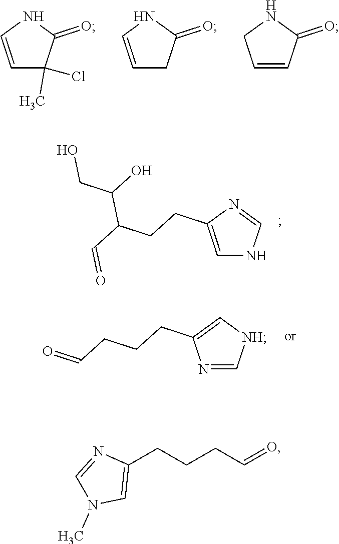
in an amount sufficient to prevent or reduce physiological glycation of the bioprosthetic heart valve.

9. The method of claim 8, wherein the bioprosthetic heart valve is located in a human.

10. The method of claim 8, wherein the bioprosthetic heart valve is treated prior to implantation in a human.

11. A method of modifying a bioprosthetic biomaterial, the method comprising contacting the bioprosthetic biomaterial with a composition comprising: 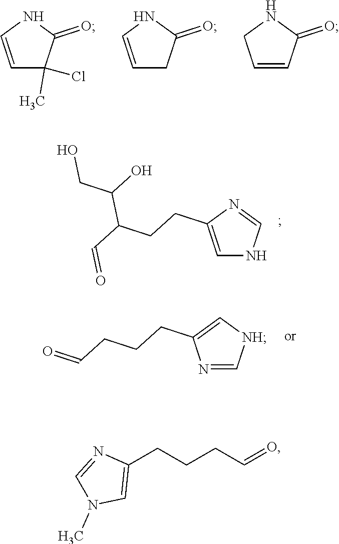
in an amount sufficient to prevent or reduce physiological glycation of the bioprosthetic biomaterial.

12. The method of claim 11, wherein the bioprosthetic biomaterial is a bioprosthetic heart valve, patch, urethral patch, aortic homografts, valve prostheses, stented valve, stentless valve, graft, dural graft, pulmonary ligament, artificial heart, or conduit.

13. The method of claim 12, wherein the bioprosthetic biomaterial is a bioprosthetic heart valve.

14. The method of claim 13, wherein the bioprosthetic heart valve is located in a human.

15. The method of claim 13, wherein the bioprosthetic heart valve is modified prior to implantation in a human.

16. A method of treating a human having a bioprosthetic heart valve to prevent or reduce physiological glycation of the bioprosthetic heart valve, the method comprising administering to the human a therapeutically effective amount of a pharmaceutical composition comprising:

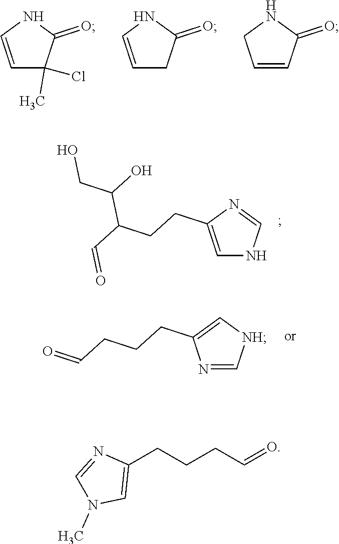

17. A bioprosthetic biomaterial treated with a composition comprising a compound that:
a. reacts directly or indirectly with glycation-susceptible sites in the bioprosthetic biomaterial to generate stable adducts that prevent the sites from reacting with physiologic glycation precursors;
b. has similar reactivity with the glycation-susceptible sites as the physiologic glycation precursors;
c. undergoes chemical rearrangements that allow for the resolution of intermediates to stable, permanent adducts that are unreactive or minimally reactive to glycation and do not follow glycation-like chemical rearrangements that result in the generation of glycation product-like moieties; and
d. is minimally deleterious or non-deleterious to the function of the bioprosthetic biomaterial,
in an amount sufficient to prevent or reduce physiologic glycation of the bioprosthetic biomaterial.

18. A method of protecting a bioprosthetic biomaterial from physiologic glycation or covalent incorporation of infiltrated circulating proteins or calcification, the method comprising extended incubation of the bioprosthetic biomaterial in water or compatible buffers, at an elevated temperature.

19. The method of claim 1, wherein the treatment renders the tissue resistant to the formation of at least one of (a) heterocyclic or cross-linking glycation products without significantly diminishing formation of linear-chain or cell-signaling glycation products or vice-versa, or (b) linear-chain or cell-signaling glycation products without significantly diminishing formation of heterocyclic or cross-linking glycation products.

20. The method of claim 19, wherein the resultant tissue is utilized as at least one of (a) a platform for decoupling the biological effects of the heterocyclic or cross-linking glycation products from those of linear-chain or cell-signaling glycation products, (b) an experimental or laboratory implement for decoupling the biological effects of the heterocyclic or cross-linking glycation products from those of linear-chain or cell-signaling glycation products, (c) a platform for the screening of therapeutic or drug compounds or interventional treatments, (d) a platform for decoupling the biological effects of the linear-chain or cell-signaling glycation products from those of heterocyclic or cross-linking glycation products, (e) an experimental or laboratory implement for decoupling the biological effects of linear-chain or cell-signaling glycation products from those of the heterocyclic or cross-linking glycation products, or (f) a platform for the screening of therapeutic or drug compounds or interventional treatments.

21. The method of claim 20, wherein the experimental or laboratory implement is cell culture incubations and chemical or biochemical analyses.

22. The method of claim 20, wherein the interventional treatments are anti-glycation interventions.
